(19) United States
(12) Patent Application Publication
(10) Pub. No.: US 2025/0221675 A1
(43) Pub. Date: Jul. 10, 2025

(54) SYSTEMS AND METHODS FOR CONDUCTING REMOTE NEURO-OPHTHALMIC EXAMINATIONS ON A MOBILE DEVICE

(71) Applicant: Thomas Jefferson University, Philadelphia, PA (US)

(72) Inventors: Nikolaos Mouchtouris, Philadelphia, PA (US); James Evans, Newtown Square, PA (US); Vadim Geyfman, Marlton, NJ (US); Stephane Krumenacker, New York, NY (US)

(21) Appl. No.: 18/853,928

(22) PCT Filed: Apr. 6, 2023
(86) PCT No.: PCT/US23/17671

§ 371 Date: Oct. 3, 2024

Related U.S. Application Data
(60) Provisional application No. 63/328,514, filed on Apr. 7, 2022.

(51) Int. Cl. A61B 5/00; A61B 3/00; A61B 3/032

(52) U.S. Cl. CPC A61B 5/7465; A61B 3/0033; A61B 3/0041; A61B 3/032; A61B 5/4064; A61B 5/6898; A61B 2562/0257

(57) ABSTRACT

One aspect of the invention provides a processor-implemented method for conducting a remote neuro-ophthalmic examination using a mobile device. The method includes: receiving, from a distance sensor of a remote device, data corresponding to a distance of a user from the mobile device; determining, from the received data, a distance of the user from the mobile device; adjusting a size parameter of a neuro-ophthalmic examination of the mobile device; and displaying the neuro-ophthalmic examination via a display of the mobile device and according to the size parameter.

### CROSS-REFERENCE TO RELATED APPLICATION

The present application claims priority to U.S. Provisional Patent Application No. 63/328,514, filed Apr. 7, 2022, which is incorporated herein by reference in its entirety.

### BACKGROUND OF THE INVENTION

Conventional medical practices are often limited to in-person meetings between a patient and a medical professional. This can be a great burden on a patient, particularly where the patient lives a significant distance away from a corresponding medical center, or if the patient's medical condition requires numerous patient-medical professional interactions.

Telemedicine offers the ability to reduce these patient burdens. However, while advances have been made in telemedicine, conventional telemedicine platforms are limited in their ability to perform certain examinations. This prevents the detailed and thorough assessment of patients. Technology that enables data-driven examination of patients via a mobile data can drastically increase the impact of telemedicine on patient care as well as clinical trials.

### SUMMARY

One aspect of the invention provides a processor-implemented method for conducting a remote neuro-ophthalmic examination using a mobile device. The method includes: receiving, from a distance sensor of a remote device, data corresponding to a distance of a user from the mobile device; determining, from the received data, the distance of the user from the mobile device; adjusting a size parameter of a neuro-ophthalmic examination of the mobile device; and displaying the neuro-ophthalmic examination via a display of the mobile device and according to the size parameter.

Another aspect of the invention provides a processor-implemented method for conducting a remote neuro-ophthalmic examination. The method includes: displaying a neuro-ophthalmic examination via a display of a mobile device; detecting, via a sensor of the mobile device; that the mobile device is repositioned with respect to a user's eyes; receiving, from a user interface of the mobile device, user input during repositioning; determining a location of the mobile device when the user input is received; and determining a location in a field of vision for the user, wherein the user input corresponds to the location of the mobile device.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a system using a mobile device for conducting remote neuro-ophthalmic examinations according to an embodiment of the present disclosure.

FIG. 2 depicts a server for conducting remote neuro-ophthalmic examinations according to an embodiment of the present disclosure.

FIGS. 3-5 depict screenshots for conducting remote neuro-ophthalmic examinations according to embodiments of the present disclosure.

FIGS. 6-8 depict process flows for conducting remote neuro-ophthalmic examinations according to embodiments of the present disclosure.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Systems, devices, and associated methods for conducting remote neuro-ophthalmic examinations are described herein. Conducting remote neuro-ophthalmic examinations has proven difficult, in part due to the technological limitations relied on for remote examinations. For example, mobile devices such as cell phones, smart watches, tablets, and the like, would be ideal for conducting neuro-ophthalmic examinations due to their ubiquitous nature. However, remote devices also include disadvantages that make conducting neuro-ophthalmic exams problematic. For example, neuro-ophthalmic exams typically require a static (or at least monitored) distance between the user's eyes and the displayed exam, and the distance between a remote device and a user may be dependent on how a user holds the device away from her face. Another disadvantage for remote devices is the relatively small display screen for displaying an exam. Some neuro-ophthalmic exams require monitoring a user's field of vision that is too large for a typical remote device to be able to display at one time.

The disclosure provided herein utilizes remote devices to effectively implement the qualitative and quantitative evaluation of visual function as well as cranial nerve functions. Such remote device-based evaluation can be utilized for telemedicine, streamlining patient care, improving clinical trials, and screening of the general population, such as for school and clearance for driving and sports. This mobile device-based evaluation can be used for the diagnosis of disease and tracking of disease progression, resolution, or recurrence. Multiple cranial nerve and neuro-ophthalmic, such as an Amsler grid, a double-vision exam, a visual acuity exam, visual field, and the like, can be implemented on a remote device, such as a cell phone, a tablet, and the like. The remote device can, in some cases, determine the distance from a user's eyes to the remote device display. Based on the distance, the remote device can adjust a size or size parameter of a neuro-ophthalmic device displayed by the remote device. In other cases, a remote device can implement a double-vision exam, which can utilize various sensors of the remote device to determine the vertical and horizontal degrees at which the user experiences double vision or distortion of an object displayed by the remote device. Further, the remote device can determine device acceleration, magnetic field, the user's facial features and eye gaze, all of which are utilized to perform the aforementioned evaluations.

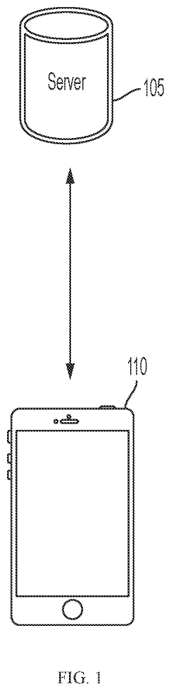

FIG. 1 depicts a system for remote neuro-ophthalmic examinations according to an embodiment of the present disclosure. The system can include a server 105 and a remote device 110.

The server 105 can store instructions for performing a remote neuro-ophthalmic examination. In some cases, the server 105 can also include a set of processors that execute the set of instructions. Further, the server 105 can be any type of server capable of storing and/or executing instructions, for example, an application server, a web server, a proxy server, a file transfer protocol (FTP) server, and the like. In some cases, the server 105 can be a part of a cloud computing architecture, such as a Software as a Service (SaaS), Development as a Service (DaaS), Data as a Service (DaaS), Platform as a Service (PaaS), and Infrastructure as a Service (IaaS).

A remote device 110 can be in electronic communication with the server 105 and can display the remote neuro-ophthalmic exam to a user. The remote device 110 can include a display for displaying the remote neuro-ophthalmic exam, and a user input device, such as a touchscreen, mouse, keyboard, or touchpad, for logging and transmitting user input corresponding to the remote neuro-ophthalmic exam. In some cases, the remote device 110 can include a set of processors for executing the remote neuro-ophthalmic exam (e.g., from instructions stored in memory). For example, in some cases the remote device 110 can download a program (e.g., from an app store) for implementing the remote neuro-ophthalmic exam, the results of which may then be transmitted to the server 105. Examples of a remote device include, but are not limited to, a cell phone, a tablet, a smartwatch, a personal digital assistant, an e-reader, a mobile gaming device, and the like.

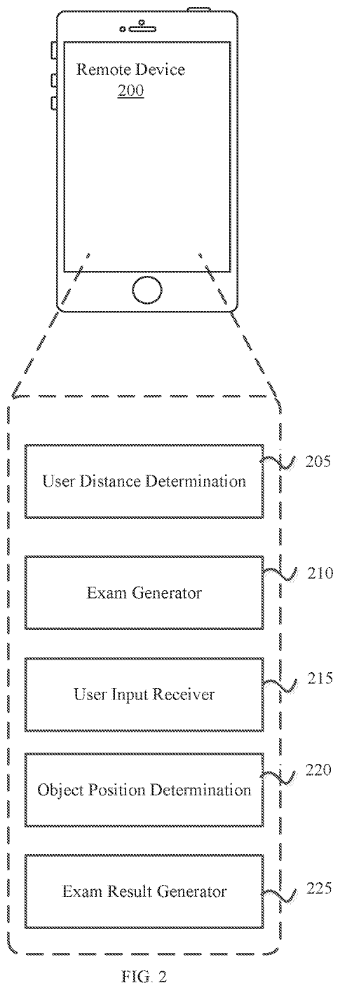

FIG. 2 depicts a remote device 200 for conducting a remote neuro-ophthalmic exam according to an embodiment of the present disclosure. The remote device can be an example of the remote device 110 as discussed with reference to FIG. 1. The remote device 200 can include a user distance determination component 205, an exam generator 210, a user input receiver 215, an object position determination component 220, and a remote neuro-ophthalmic result determination component 225 (e.g., exam result generator).

### Neuro-Ophthalmic Examination Size

The user distance determination component 205 can detect how far away a user's eyes are from the display screen of the remote device. For example, the display can provide directions to a user to hold the display a distance further away from the user's face. The remote device can activate a sensor (e.g., an infrared camera, a camera, and the like), and can measure the distance the user's face is from the display screen. In some cases, processors of the remote device can measure a distance separating various aspects of the user's face for determining how far away from the device the user's face is. For example, from a captured image of the user's face, the processors can measure the distance between the user's eyes, which can be indicative of how far the user's face is from the display (e.g., the distance between eyes can be generally uniform throughout a population, and the smaller the distance perceived by the camera, the farther away the user's face is from the display).

The exam generator 210 can then generate a neuro-ophthalmic exam from the determined distance away. For example, neuro-ophthalmic exams that can be displayed by the remote device can include a Snellen test, an Amsler grid, a double-vision test, and the like. A set size of the exam can be stored by the remote device, for example, as a pixel size for the given size of the display. The remote device can also store a standard distance away from the screen for the user and for the given exam. For example, a standard size (e.g., 100%) can be stored for the exam to be displayed at particular distance away from the user (e.g., 5 ft). However, based on the determined distance away from the display the user is, the exam generator 210 can adjust the size of the exam displayed. For example, if the standard distance away for a user is 5 ft, but the user distance determination component 205 determines the user is 6 ft away, the exam generator may adjust the size of the exam displayed to be larger (e.g., approximately a 20% increase).

In some cases, the exam generator 210 can adjust a size of a given portion of an exam. For example, the exam to be displayed may include typing. The exam generator 210 may adjust the font size of the exam based on the distance away from the display the user is. In other examples, the exam to be displayed may include different animated objects, the sizing of which may be adjusted based on the user distance away.

### Neuro-Ophthalmic Exam Implementation

In some cases, the exam generator 210 can generate a repositionable animated object for the display screen of the remote device (e.g., remote device 110/200), for example for a responsive visual field test. The repositionable animated object can be any number of objects having a defined body, which includes but is not limited to a dot, a circle, a triangle, a star, a rectangle, an ellipse, and the like. Further, the object generator 210 can reposition the animated object on the display screen over a period of time. For example, the animated object can move in a predefined direction at a predefined speed across the display upon initiation of the double vision procedure. In some cases, the object generator can also generate a reference point to be displayed by the display. The reference point may be a stationary object displayed on the screen. In some cases, the animated object may move in relation to the reference point, for example moving away from, or towards the reference point.

In some other cases, the exam generator 210 can generate a segment of an Amsler grid. For example, the segment can include a number of grid segments (e.g., squares) that can be sized according to the distance away the user is from the display. The portion can also include, in some cases, less than the full Amsler grid that is to be displayed.

The exam generator 210 can also update the exam displayed as the remote device repositions with respect to the user eyes. For example, the remote device may utilize sensors capable of tracking the location of the remote device with respect to the user. For example, the remote device may utilize an accelerometer, gyroscope, or magnetometer of the remote device to determine a location (e.g., via pitch parameters, roll parameters, gravity, magnetic field, high-definition camera footage, and the like) of the remote device with respect to an original position of the remote device (e.g., when the exam initiated). Facial recognition and gaze tracking functionality and components of the mobile device, which may identify eyes, mouth, nose, chin as well as direction of gaze, can also be used to track the location of the mobile device relative to the user, to detect a neurological deficit. Based on the updated location of the remote device, the exam generator 210 can adjust the exam displayed. For example, in the Amsler grid scenario above, another segment of the grid may be shown based on the updated location of the phone. For example, if a user tilts the phone higher up, the remote device sensors can determine the new, higher location of the remote device, and thus display grid segments of the upper segment of the Amsler grid. In the case of the double-vision or other responsive visual field tests, the generated objects may be static on the display itself, even when the remote device is repositioned relative to the user.

The user input receiver 215 can receive user input from the computing device. For example, the user input can be a mouse click, a keyboard click, a touch on a touchpad or mobile device screen, and the like. The user input receiver 215 can receive the user input and log different parameters of the user input. For example, the user input receiver 215 can identify a timestamp of the user input, the type of user input (e.g., mouse click, keyboard click, etc.) and the like. The remote device 200 can store the user input in memory.

The object distance determination component 220 can determine a location the received input corresponds to with respect to the users view range. For example, in the case of an Amsler grid exam, the object distance determination component 220 can identify a grid section user input is received for (e.g., via a touchscreen), a particular section of the Amsler grid being displayed when the input is received, or both. In the case of a double vision exam or a responsive visual field test, the object distance determination component 220 can determine a distance or angle from center of a user's viewpoint when the user input is received. The determination can be based on a timestamp of the received user input. In some cases, the determination can be based on the speed the remote device is repositioned, the direction in which the remote device is repositioned, and/or an initiation timestamp corresponding to when the exam began (e.g., when the exam is initially displayed).

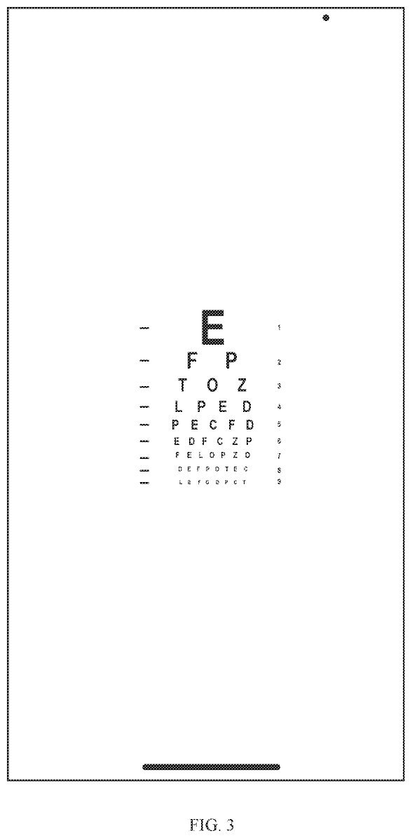

FIG. 3 depicts a screenshot of a Snellen eye examination according to embodiments of the present disclosure. The chart may be displayed on the display of a remote device, and may be resized based on the determined distance away from the user's eyes. In some cases, the font size of the Snellen chart may be resized based on the determined distance. In other cases, the letters or symbols displayed may be switched based on the user's response. The font color and display light intensity may be varied based on the user's performance.

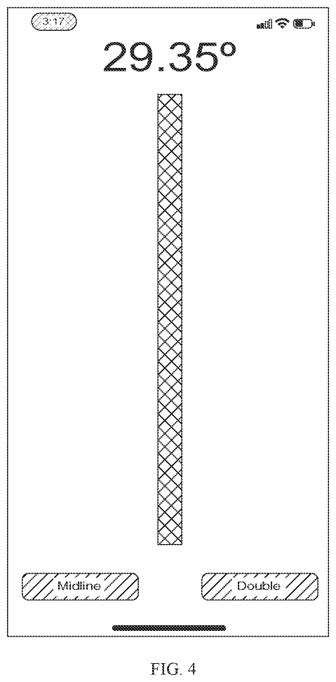

FIG. 4 depicts a screenshot of a double vision procedure according to embodiments of the present disclosure. In this embodiment, a vertical bar may be statically positioned on the display screen of the remote device. The user may be instructed to reposition the display with respect to the user's center line of vision (e.g., keeping the user's eye position focused at the display). As the remote device is repositioned, the user may provide input (e.g., through the touchscreen) when the user experiences double vision of the vertical bar. When the user input is received, the remote device can determine an angle from the center line of vision of the user the remote device (the vertical bar) is located. The same process can be repeated for vertical double vision using a horizontal bar.

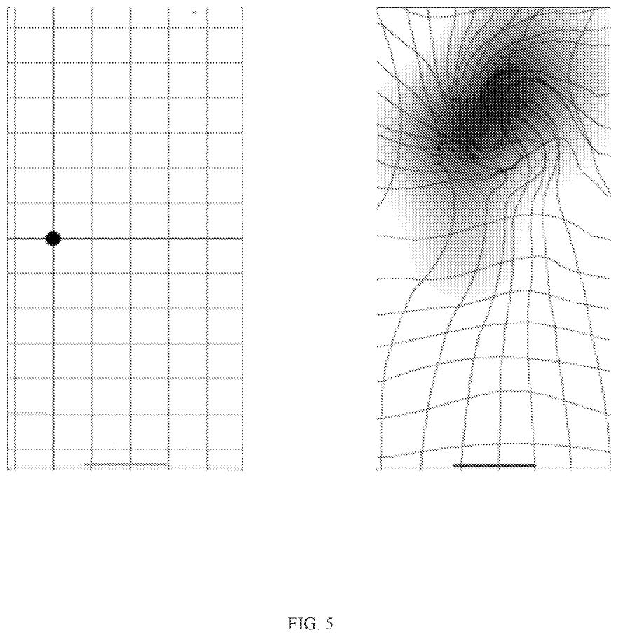

FIG. 5 depicts an Amsler grid exam according to an embodiment of the present disclosure. A segment of the Amsler grid can be initially displayed on the display screen of the remote device. The user can then be instructed to reposition the remote device with respect to the user's center view line. As the remote device is repositioned, the remote device may display different segments of the Amsler grid (based on the remote device determining new locations of the remote device with respect to the user's eyes). The user can reposition the remote device to visualize the different segments of a virtual Amsler grid that is displayed through the device. If a distortion is noted in any segment of the grid, the user provides input. The remote device determines its location with respect to the user and thereby calculates the location of the distortion. The user can also be instructed to provide input (e.g., through the touchscreen) when the user experiences distortion in the Amsler grid. When the user input is received, the remote device can determine a location or angle where the distortion occurs based on the location of the remote device at the time the user input is received.

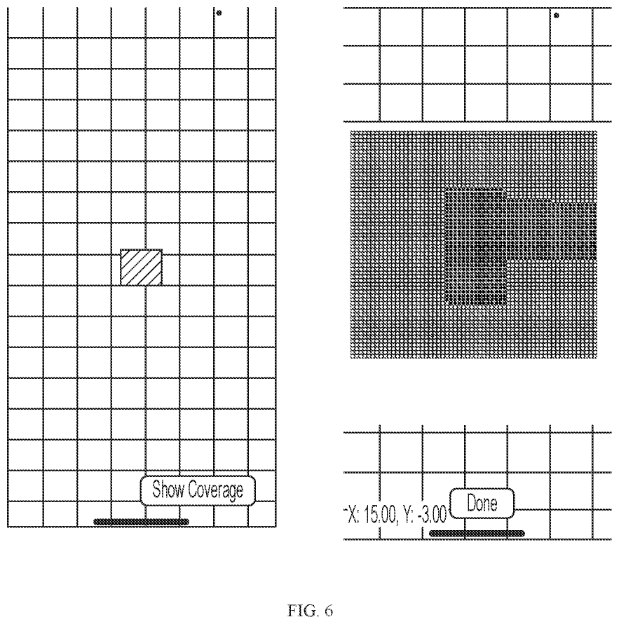

FIG. 6 depicts images for a responsive visual field test according to an embodiment of the present disclosure. The dimensions of responsive visual field vary as the user moves the remote device towards or away from the user's face. Using the multi-sensor data described in this disclosure, the processor determines the location of a displayed image in the virtual visual field and correlates to the user's true visual field. The left image depicts an image statically displayed on the display of the remote device. The position of the image is calculated based on the position of the remote device with respect to the user. The user can be instructed to move the remote device with respect to the user's center line of vision. The user can be instructed to provide user input when the user fails to see the image of the display screen, as the user repositions the remote device. The right image depicts a path the remote device took with respect to the user's center line of vision. When the user provides input by touching the screen or clicking a button to indicate that the user fails to see the image, the position of that point in space is recorded on the peripheral vision map seen in FIG. 6. Based on the displayed map, the user is instructed to move the mobile device to areas of the visual field that have not yet been tested until the user has explored the entirety of their true visual field through the mobile device.

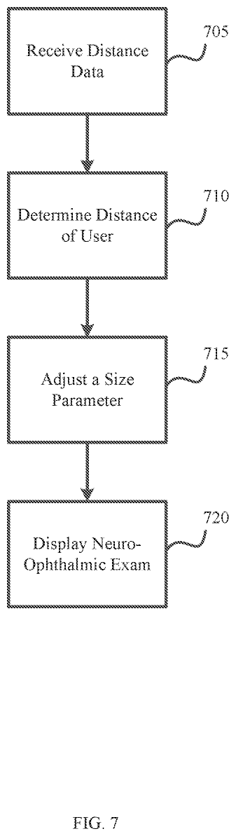

FIG. 7 depicts a process flow for conducting a neuro-ophthalmic examination according to an embodiment of the present disclosure. The process flow can be implemented by the system (including server 105 and remote device 110) of FIG. 1. In some cases, the process flow can be implemented by remote device 110 of FIG. 1.

At Step 705, a distance sensor of a remote device can receive data corresponding to a distance of a user from the remote device. The display can be of a remote device, such as remote device 110 of FIG. 1.

At Step 710, a distance of the user from the remote device can be determined from the received data.

At Step 715, a size parameter of a neuro-ophthalmic examination of the remote device can be adjusted based on the received data.

At Step 720, the neuro-ophthalmic examination can be displayed via a display of the remote device and according to the size parameter.

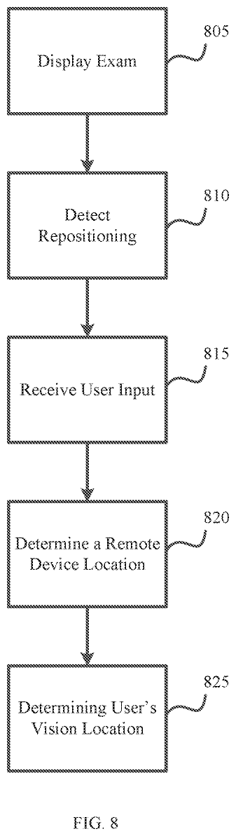

FIG. 8 depicts a process flow for conducting a neuro-ophthalmic examination according to an embodiment of the present disclosure. The process flow can be implemented by system (including server 105 and remote device 110) of FIG. 1. In some cases, the process flow can be implemented by remote device 110 of FIG. 1.

At Step 805, a neuro-ophthalmic examination can be displayed via a display of a remote device.

At Step 810, a sensor of the remote device can detect that the remote device is repositioned with respect to a user's eyes.

At Step 815, a user interface of the remote device can receive user input during the repositioning.

At Step 820, a location of the remote device can be determined when the user input is received.

At Step 825, a location in a field of vision for the user is determined, wherein the user input corresponds to (and/or can be determined from) the location of the remote device.

### Equivalents

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A processor-implemented method for conducting a remote neuro-ophthalmic examination using a mobile device, comprising:
receiving, from a distance sensor of a remote device, data corresponding to a distance of a user from the mobile device;
determining, from the received data, the distance of the user from the mobile device;
adjusting a size parameter of a neuro-ophthalmic examination of the mobile device; and
displaying the neuro-ophthalmic examination via a display of the mobile device and according to the size parameter.

2. The processor-implemented method of claim 1, further comprising:
receiving user input via a user interface during the displaying of the neuro-ophthalmic examination.

3. The processor-implemented method of claim 2, further comprising:
generating a neuro-ophthalmic examination report for the user based on the displayed neuro-ophthalmic examination and the received user input.

4. The processor-implemented method of claim 1, wherein the size parameter comprises a total size of the neuro-ophthalmic examination, a font size of the neuro-ophthalmic examination, or a combination thereof.

5. The processor-implemented method of claim 1, wherein the neuro-ophthalmic examination comprises a Snellen test, an Amsler grid, or a double-vision examination.

6. A processor-implemented method for conducting a remote neuro-ophthalmic examination, comprising:
displaying a neuro-ophthalmic examination via a display of a mobile device;
detecting, via a sensor of the mobile device, that the mobile device is repositioned with respect to a user's eyes;
receiving, from a user interface of the mobile device, user input during repositioning;
determining a location of the mobile device when the user input is received; and
determining a location in a field of vision for the user, wherein the user input corresponds to the location of the mobile device.

7. The processor-implemented method of claim 6, wherein the neuro-ophthalmic examination comprises an Amsler grid.

8. The processor-implemented method of claim 7, further comprising:
identifying a location on the Amsler grid based on the repositioning and the user input received; and
determining the user experiences a visual distortion at the location on the Amsler grid.

9. The processor-implemented method of claim 7, further comprising:
determining a direction of movement and a distance of movement during the repositioning; and
displaying a different segment of the Amsler grid on the display of the mobile device based on the direction of movement and the distance of movement.

10. The processor-implemented method of claim 6, wherein the neuro-ophthalmic examination comprises a double-vision test.

11. The processor-implemented method of claim 10, further comprising:
identifying a location of a symbol on the display and in relation to the user's eyes based on the positioning and the user input received; and
determining an angle of a viewing range of the user that the user experiences double vision based on the location of the symbol.

12. The processor-implemented method of claim 6, wherein the neuro-ophthalmic examination comprises a responsive visual field test.
